(19) United States
(12) Patent Application Publication
(10) Pub. No.: US 2002/0111290 A1
(43) Pub. Date: Aug. 15, 2002

(54) Uses of mammalian genes and related reagents

(76) Inventors: Bernhard Homey, Dusseldorf (DE); Monica Zepeda, San Diego, CA (US); Albert Zlotnik, Palo Alto, CA (US)

Correspondence Address: DNAX RESEARCH INSTITUTE, LEGAL DEPARTMENT, 901 CALIFORNIA AVENUE, PALO ALTO, CA, 94304, US

(21) Appl. No.: 09/995,534

(22) Filed: Nov. 27, 2001

Related U.S. Application Data
(60) Provisional application No. 60/250,782, filed on Dec. 1, 2000.

(51) Int. Cl. A61K 31/00; C12Q 1/68; G01N 33/53

(52) U.S. Cl. 514/1; 514/12; 435/6; 435/7.1

(57) ABSTRACT

Methods for treating, diagnosing, or evaluating various medical conditions. Correlations of chemokine or receptor expression with medical status are provided.

This application claims benefit of U.S. Provisional Patent Application No. 60/250,782, filed Dec. 1, 2000.

### FIELD OF THE INVENTION

The present invention relates generally to uses of mammalian genes and related reagents. More specifically, the invention relates to identification of mammalian genes whose expression levels are implicated in medical conditions affecting skin or wound healing, e.g., inflammatory skin conditions. Diagnostic and therapeutic uses result.

### BACKGROUND OF THE INVENTION

Because inflammatory responses are often mediated by cytokine or chemokine activity, methods to evaluate synthesis of these signaling molecules would be advantageous for diagnosis of selected diseases. The present invention relates generally to identification of genes which may directly be of use to treat, or alternatively, to evaluate status of medical conditions affecting skin or wound healing. See, e.g., Fitzpatrick, et al. (eds. 1993) Dermatology in General Medicine 4th ed., McGraw-Hill, NY; Bos (ed. 1989) Skin Immune System CRC Press, Boca Raton, Fla.; Callen (1996) General Practice Dermatology Appleton and Lange; Rook, et al. (eds. 1998) Textbook of Dermatology Blackwell; Habifor and Habie (1995) Clinical Dermatology: A Color Guide to Diagnosis and Therapy Mosby; and Grob (ed. 1997) Epidemiology, Causes and Prevention of Skin Diseases Blackwell; Hess and Salcido (2000) Wound Care Springhouse Pub Co (ISBN: 1582550549); Mani, et al. (1999) Chronic Wound Healing: Clinical Measurement and Basic (ISBN: 0702022063); Wyngaarden and Smith (eds.) Cecil's Textbook of Medicine (W. B. Saunders Co. 1985); Berkow (ed.) The Merck Manual of Diagnosis and Therapy (Merck Sharp & Dohme Research Laboratories, 1982); and Harrison's Principles of Internal Medicine, 12th Ed., McGraw-Hill, Inc. (1991), all of which are incorporated herein by reference.

Problems with skin surfaces or wounds can be seriously irritating or disfiguring, and eventually may lead to more serious complications. Thus, a need exists for effective treatment, both prophylactic and curative, to alleviate the symptoms of those conditions. Alternatively, methods of diagnosis, e.g., of abnormal or modified health of those tissues will be useful. The present invention provides both.

### SUMMARY OF THE INVENTION

The present invention is based, in part, upon the recognition of the correlation of chemokine and chemokine receptor agonists and antagonists in skin inflammation disorders, and in wound healing.

The present invention provides methods of diagnosing or evaluating a skin injury or condition affecting the skin, the method comprising evaluating expression of: a chemokine selected from MCP-2 (CCL8), DC-CK1 (CCL18), TARC (CCL17), RANTES (CCL5), MIP3b (CCL19), I-309 (CCL1), MIG (CXCL9), IP-10 (CXCL10), ITAC (CXCL11), BCA-1 (CXCL13), lymphotactin (XCL1), MDC (CCL22), IL-8 (CXCL8), MCP-3 (CCL7), MCP-1 (CCL2), or SDF-1; or a chemokine receptor selected from CCR5, CCR7, CXCR3, CXCR5, XCR1, CCR2, CCR4, CCR8, or CXCR4. Typically, the condition is selected from lupus erythematosus, atopic dermatitis, cutaneous wound, skin healing, or an inflammatory condition; or the evaluating is: measuring a plurality of the expression levels; measuring mRNA levels; or measuring protein levels.

The invention further provides methods of treating a condition affecting the skin, the method comprising administering an antagonist of: a chemokine selected from MCP-2 (CCL8), DC-CK1 (CCL18), TARC (CCL17), RANTES (CCL5), MIP3b (CCL19), I-309 (CCL1), MIG (CCL9), IP-10 (CXCL10), ITAC (CXCL11), BCA-1 (CXCL13), lymphotactin (XCL1), MDC (CCL22), IL-8 (CXCL8), MCP-3 (CCL7), or MCP-1 (CCL2); or a chemokine receptor selected from CCR5, CCR7, CXCR3, CXCR5, XCR1, CCR2, CCR4, CCR8, or CXCR4. Typically, the administering is: a plurality of the antagonists; or in combination with another therapeutic. Often, the antagonist is an antibody which prevents interaction of: the chemokine with its receptor, or the chemokine receptor with its ligand; or the treating is preventative.

In various embodiments, the condition is lupus erythematosus, and the antagonist is of: a chemokine selected from MCP-2 (CCL8), RANTES (CCL5), MIP3b (CCL19), MIG (CXCL9), IP-10 (CXCL10); ITAC (CXCL11); BCA-1 (CXCL13), or lymphotactin (XCL1); or a chemokine receptor selected from CCR5, CCR7, CXCR3, CXCR5, or XCR1. In other embodiments, the condition is atopic dermatitis, and the antagonist is of: a chemokine selected from DC-CK1 (CCL18), TARC (CCL17), I-309 (CCL1), MDC (CCL22), IP-10, MIG, or ITAC; or a CCR2, CXCR3, CCR4, or CCR8 chemokine receptor.

In addition, the invention provides methods of accelerating wound healing comprising administering to an individual suffering from a wound a chemokine selected from lymphotactin (XCL1), IL-8 (CXCL8), MCP3 (CCL7), MCP1 (CCL2), MCP-2 (CCL8), RANTES (CCL5), MIG (CXCL9), or SDF-1. In some embodiments, the administering is: a plurality of the chemokines; in combination with another therapeutic; or by expression of a nucleic acid. Often, the healing is from skin loss from burn.

### DETAILED DESCRIPTION OF THE PREFERRED EMBODIMENTS

OUTLINE

I. General

A. Inflammatory Skin Diseases and Conditions

B. Wound healing

C. Chemokines and Receptors

II. Antagonists and Agonists

A. Blocking ligand

B. Blocking receptor

C. Agonists

III. Diagnostic uses; Therapeutic Compositions, Methods

A. indications

B. combination compositions

C. unit dose

D. administration

I. General

The skin is an important boundary which separates the organism from its environment, including hostile organisms and antigens. Thus, the processes occurring at the skin are important. Inflammatory skin effects can be greatly discomforting, or may lead to significant medical problems.

Likewise, wound healing is an important process involving repair of skin or internal organs. The rate of healing or a wound may be regulated by, or affected by, the presence of particular chemokines or chemokine receptors.

The present invention resulted from studies directed to whether modified expression of chemokines or chemokine receptors correlated with conditions affecting, e.g., skin inflammation or wound healing. Increased expression of chemokines could result in recruitment of inflammatory cells, e.g., macrophages, dendritic cells, or lymphocytes, and which may contribute to lesion development in skin inflammation and related conditions. These included several chemokines and chemokine receptors.

II. Antagonists and agonists

Blockage of the signaling pathway can be achieved by antagonists of the chemokine, e.g., antibodies to the ligand, antibodies to the receptor, etc. Interference with the ligand-receptor interaction has proven to be an effective strategy for the development of antagonists.

There are various means to antagonize the signaling mediated by ligand. Two apparent means are to block the ligand with antibodies; a second is to block the receptor with antibodies. Various epitopes will exist on each which will block their interaction, e.g., causing steric hindrance blocking interaction. The correlation of ability to block signaling would not necessarily be expected to correlate with binding affinity to either ligand or receptors. Another means is to use a ligand mutein which retains receptor binding activity, but fails to induce receptor signaling. The mutein may be a competitive inhibitor of signaling ligand.

Alternatively, small molecule libraries may be screened for compounds which may block the interaction or signaling mediated by an identified ligand-receptor pairing.

The present invention provides for the use of an antibody or binding composition which specifically binds to a specified chemokine ligand, preferably mammalian, e.g., primate, human, cat, dog, rat, or mouse. Antibodies can be raised to various chemokine proteins, including individual, polymorphic, allelic, strain, or species variants, and fragments thereof, both in their naturally occurring (full-length) forms or in their recombinant forms. Additionally, antibodies can be raised to receptor proteins in both their native (or active) forms or in their inactive, e.g., denatured, forms. Anti-idiotypic antibodies may also be used.

A number of immunogens may be selected to produce antibodies specifically reactive with ligand or receptor proteins. Recombinant protein is a preferred immunogen for the production of monoclonal or polyclonal antibodies. Naturally occurring protein, from appropriate sources, e.g., primate, rodent, etc., may also be used either in pure or impure form. Synthetic peptides, made using the appropriate protein sequences, may also be used as an immunogen for the production of antibodies. Recombinant protein can be expressed and purified in eukaryotic or prokaryotic cells as described, e.g., in Coligan, et al. (eds. 1995 and periodic supplements) Current Protocols in Protein Science John Wiley & Sons, New York, N.Y.; and Ausubel, et al (eds. 1987 and periodic supplements) Current Protocols in Molecular Biology, Greene/Wiley, New York, N.Y. Naturally folded or denatured material can be used, as appropriate, for producing antibodies. Either monoclonal or polyclonal antibodies may be generated, e.g., for subsequent use in immunoassays to measure the protein, or for immunopurification methods.

Methods of producing polyclonal antibodies are well known to those of skill in the art. Typically, an immunogen, preferably a purified protein, is mixed with an adjuvant and animals are immunized with the mixture. The animal's immune response to the immunogen preparation is monitored by taking test bleeds and determining the titer of reactivity to the protein of interest. For example, when appropriately high titers of antibody to the immunogen are obtained, usually after repeated immunizations, blood is collected from the animal and antisera are prepared. Further fractionation of the antisera to enrich for antibodies reactive to the protein can be performed if desired. See, e.g., Harlow and Lane; or Coligan. Immunization can also be performed through other methods, e.g., DNA vector immunization. See, e.g., Wang, et al. (1997) Virology 228:278-284.

Monoclonal antibodies may be obtained by various techniques familiar to researchers skilled in the art. Typically, spleen cells from an animal immunized with a desired antigen are immortalized, commonly by fusion with a myeloma cell. See, Kohler and Milstein (1976) Eur. J. Immunol. 6:511-519. Alternative methods of immortalization include transformation with Epstein Barr Virus, oncogenes, or retroviruses, or other methods known in the art. See, e.g., Doyle, et al. (eds. 1994 and periodic supplements) Cell and Tissue Culture: Laboratory Procedures, John Wiley and Sons, New York, N.Y. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. Alternatively, one may isolate DNA sequences which encode a monoclonal antibody or a binding fragment thereof by screening a DNA library from human B cells according, e.g., to the general protocol outlined by Huse, et al. (1989) Science 246:1275-1281.

Antibodies or binding compositions, including binding fragments and single chain versions, against predetermined fragments of ligand or receptor proteins can be raised by immunization of animals with conjugates of the fragments with carrier proteins. Monoclonal antibodies are prepared from cells secreting the desired antibody. These antibodies can be screened for binding to normal or defective protein. These monoclonal antibodies will usually bind with at least a K_{D} of about 1 mM, more usually at least about 300 μM, typically at least about 10 μM, more typically at least about 30 μM, preferably at least about 10 μM, and more preferably at least about 3 μM or better.

In some instances, it is desirable to prepare monoclonal antibodies (mAbs) from various mammalian hosts, such as mice, rodents, primates, humans, etc. Description of techniques for preparing such monoclonal antibodies may be found in, e.g., Stites, et al. (eds.) Basic and Clinical Immunology (4th ed.) Lange Medical Publications, Los Altos, Calif., and references cited therein; Harlow and Lane (1988) Antibodies: A Laboratory Manual CSH Press; Goding (1986) Monoclonal Antibodies: Principles and Practice (2d ed.) Academic Press, New York, N.Y.; and particularly in Kohler and Milstein (1975) Nature 256:495-497, which discusses one method of generating monoclonal antibodies. Summarized briefly, this method involves injecting an animal with an immunogen. The animal is then sacrificed and cells taken from its spleen, which are then fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Other suitable techniques involve selection of libraries of antibodies in phage or similar vectors. See, e.g., Huse, et al. (1989) “Generation of a Large Combinatorial Library of the Immunoglobulin Repertoire in Phage Lambda,” Science 246:1275-1281; and Ward, et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see, Cabilly, U.S. Pat. No. 4,816,567; and Queen, et al. (1989) Proc. Nat'l Acad. Sci. USA 86:10029-10033; or made in transgenic mice, see Mendez, et al. (1997) Nature Genetics 15:146-156; also see Abgenix and Medarex technologies.

Antibodies are merely one form of specific binding compositions. Other binding compositions, which will often have similar uses, include molecules that bind with specificity to ligand or receptor, e.g., in a binding partner-binding partner fashion, an antibody-antigen interaction, or in a natural physiologically relevant protein-protein interaction, either covalent or non-covalent, e.g., proteins which specifically associate with desired protein. The molecule may be a polymer, or chemical reagent. A functional analog may be a protein with structural modifications, or may be a structurally unrelated molecule, e.g., which has a molecular shape which interacts with the appropriate binding determinants. Antibody binding compounds, including binding fragments, of this invention can have significant diagnostic or therapeutic value. They can be useful as non-neutralizing binding compounds and can be coupled to toxins or radionuclides so that when the binding compound binds to the antigen, a cell expressing it, e.g., on its surface, is killed. Further, these binding compounds can be conjugated to drugs or other therapeutic agents, either directly or indirectly by means of a linker, and may effect drug targeting.

Agonists include the chemokine proteins identified. See, e.g., GenBank and other public sequence databases. Proteins of those sequences, or variants thereof, will be used to induce receptor signaling.

III. Diagnostic Uses; Therapeutic Compositions, Methods

The invention provides means to address various skin conditions, e.g., with symptoms of inflammation. The etiology and pathogenesis are often not well understood, but they cause significant discomfort or morbidity in patients. Changes in cell migration and chemokine production appear to correlate certain skin related conditions.

Collectively these studies suggest that antagonizing these chemokines or their receptors, with the appropriate entity may offer a therapeutic modality in skin conditions or diseases, e.g., inflammatory conditions.

Diagnostic methods include such aspects as prediction of prognosis; definition of subsets of patients who will either respond or not respond to a particular therapeutic course; diagnosis of skin diseases or subtypes of conditions or diseases; or assessing response to therapy. For example, subtypes of inflammatory diseases my be defined molecularly by the comparative expression levels of a chemokine selected from MCP-2 (CCL8), DC-CK1 (CCL18), TARC (CCL17), RANTES (CCL5), MIP3b (CCL19), I-309 (CCL1), MIG (CXCL9), IP-10 (CXCL10), ITAC (CXCL11), BCA-1 (CXCL13), lymphotactin (XCL1), MDC (CCL22), IL-8 (CXCL8), MCP-3 (CCL7), or MCP-1 (CCL2); or a chemokine receptor selected from CCR5, CCR7, CXCR3, CXCR5, XCR1, or CCR2; or various combinations thereof.

Antagonists to chemokine mediated signaling have been implicated in a manner suggesting significant therapeutic effects, e.g., to decrease or prevent occurrence of symptoms. Small molecule antagonists for 7 transmembrane receptors and chemokine receptors are well known. Pertussis toxin can block the interaction of such receptors with the associated signaling G-protein coupled receptors.

The antagonists and/or agonists of the present invention can be administered alone or in combination with another inhibitor or agonist of the same or accompanying pathway; or other compounds used for the treatment of symptoms, e.g., antagonists, or steroids such as glucocorticoids.

Certain antagonists or agonists may be useful in the wound healing context to slow down the process. Thus, problems of keloid healing or hypertrophic scars may be advantageously treated from slowing down the wound healing process. Conversely, it may be desired to increase the speed of healing in, e.g., chronic ulcera or chronic wounds. This may be effected by either direct protein administration, or perhaps using a gene therapy strategy. Antagonism may be effected, e.g., by antisense treatment, antibodies, or other suppression of chemokine effects. Non cutaneous wound healing may also be targets for treatment, e.g., in abdominal or other surgeries, cartilage or joint surgeries, oral surgery, and many injuries involving stromal tissue. See, e.g., Townsend (ed. 2001) Sabiston Textbook of Surgery: The Biological Basis of Modern Surgical Practice Saunders (ISBN: 0721682693); Sabiston and Lyerly (eds. 1997) Textbook of Surgery: the Biological Basis of Modern Surgical Practice Saunders (ISBN: 0721658873); Morris and Malt (eds. 1994) Oxford Textbook of Surgery Oxford Univ. Press (ISBN: 0192618008); and Clunie (ed. 1997) Textbook of Surgery Blackwell (ISBN: 0867933534). Timing and sequence of cascades of various chemokines suggest that these processes reflect temporal series of events of infiltration of various cell types.

To prepare pharmaceutical or sterile compositions including the antibody, binding composition thereof, chemokine agonist, or small molecule antagonist, the entity is admixed with a pharmaceutically acceptable carrier or excipient which is preferably inert. Preparation of such pharmaceutical compositions is known in the art, see, e.g., Remington's Pharmaceutical Sciences and U.S. Pharmacopeia: National Formulary, Mack Publishing Company, Easton, Pa. (1984).

Antibodies, binding compositions, or chemokines are normally administered parentally, preferably intravenously. Since such proteins or peptides may be immunogenic they are preferably administered slowly, either by a conventional IV administration set or from a subcutaneous depot, e.g. as taught by Tomasi, et al, U.S. Pat. No. 4,732,863. Means to minimize immunological reactions may be applied. Small molecule entities may be orally active.

When administered parenterally the biologics will be formulated in a unit dosage injectable form (solution, suspension, emulsion) in association with a pharmaceutically acceptable parenteral vehicle. Such vehicles are typically inherently nontoxic and nontherapeutic. The therapeutic may be administered in aqueous vehicles such as water, saline, or buffered vehicles with or without various additives and/or diluting agents. Alternatively, a suspension, such as a zinc suspension, can be prepared to include the peptide. Such a suspension can be useful for subcutaneous (SQ) or intramuscular (IM) injection. The proportion of biologic and additive can be varied over a broad range so long as both are present in effective amounts. The antibody is preferably formulated in purified form substantially free of aggregates, other proteins, endotoxins, and the like, at concentrations of about 5 to 30 mg/ml, preferably 10 to 20 mg/ml. Preferably, the endotoxin levels are less than 2.5 EU/ml. See, e.g., Avis, et al. (eds.)(1993) Pharmaceutical Dosage Forms: Parenteral Medications 2d ed., Dekker, N.Y.; Lieberman, et al. (eds. 1990) Pharmaceutical Dosage Forms: Tablets 2d ed., Dekker, N.Y.; Lieberman, et al. (eds. 1990) Pharmaceutical Dosage Forms: Disperse Systems Dekker, N.Y.; Fodor, et al. (1991) Science 251:767-773, Coligan (ed.) Current Protocols in Immunology Hood, et al. Immunology Benjamin/Cummings; Paul (ed.) Fundamental Immunology; Academic Press; Parce, et al. (1989) Science 246:243-247; Owicki, et al. (1990) Proc. Nat'l Acad. Sci. USA 87:4007-4011; and Blundell and Johnson (1976) Protein Crystallography, Academic Press, New York.

Selecting an administration regimen for a therapeutic depends on several factors, including the serum or tissue turnover rate of the entity, the level of symptoms, the immunogenicity of the entity, and the accessibility of the target cells, timing of administration, etc. Preferably, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate antibody doses is found in, e.g. Bach et al., chapter 22, in Ferrone, et al. (eds. 1985) Handbook of Monoclonal Antibodies Noges Publications, Park Ridge, N.J.; and Haber, et al. (eds.) (1977) Antibodies in Human Diagnosis and Therapy, Raven Press, New York, N.Y. (Russell, pgs. 303-357, and Smith, et al., pgs. 365-389). Alternatively, doses of chemokine or small molecules are determined using standard methodologies.

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced. Preferably, a biologic that will be used is derived from the same species as the animal targeted for treatment, thereby minimizing a humoral response to the reagent.

The total weekly dose ranges for antibodies or fragments thereof, which specifically bind to ligand or receptor range generally from about 10 μg, more generally from about 100 μg, typically from about 500 μg, more typically from about 1000 μg, preferably from about 5 mg, and more preferably from about 10 mg per kilogram body weight. Generally the range will be less than 100 mg, preferably less than about 50 mg, and more preferably less than about 25 mg per kilogram body weight. Agonist or small molecule therapeutics may be used at similar molarities.

The weekly dose ranges for antagonists of chemokine receptor mediated signaling, e.g., antibody or binding fragments, range from about 1 μg, preferably at least about 5 μg, and more preferably at least about 10 μg per kilogram of body weight. Generally, the range will be less than about 1000 μg, preferably less than about 500 μg, and more preferably less than about 100 μg per kilogram of body weight. Dosages are on a schedule which effects the desired treatment and can be periodic over shorter or longer term. In general, ranges will be from at least about 10 μg to about 50 mg, preferably about 100 μg to about 10 mg per kilogram body weight. Chemokine agonists or small molecule therapeutics will typically be used at similar molar amounts, but because they likely have smaller molecular weights, will have lesser weight doses.

The present invention also provides for administration of biologics in combination with known therapies, e.g., steroids, particularly glucocorticoids, which alleviate the symptoms, e.g., associated with inflammation, or antibiotics or anti-infectives. Daily dosages for glucocorticoids will range from at least about 1 mg, generally at least about 2 mg, and preferably at least about 5 mg per day. Generally, the dosage will be less than about 100 mg, typically less than about 50 mg, preferably less than about 20 mg, and more preferably at least about 10 mg per day. In general, the ranges will be from at least about 1 mg to about 100 mg, preferably from about 2 mg to 50 mg per day. Suitable dose combinations with antibiotics, anti-infectives, or anti-inflammatories are also known.

The phrase “effective amount” means an amount sufficient to ameliorate a symptom or sign of the medical condition. Typical mammalian hosts will include mice, rats, cats, dogs, and primates, including humans. An effective amount for a particular patient may vary depending on factors such as the condition being treated, the overall health of the patient, the method route and dose of administration and the severity of side affects. When in combination, an effective amount is in ratio to a combination of components and the effect is not limited to individual components alone An effective amount of therapeutic will decrease the symptoms typically by at least about 10%; usually by at least about 20%; preferably at least about 30%; or more preferably at least about 50%. The present invention provides reagents which will find use in therapeutic applications as described elsewhere herein, e.g., in the general description for treating disorders associated with the indications described, e.g., inflammatory conditions, chronic or acute, wound healing, etc. See, e.g., Dayer (1999) J. Clin. Invest. 104:1337-1339; Gracie, et al. (1999) J. Clin. Invest. 104:1393-1401; Berkow (ed.) The Merck Manual of Diagnosis and Therapy, Merck & Co., Rahway, N.J.; Thorn, et al. Harrison's Principles of Internal Medicine, McGraw-Hill, NY; Gilman, et al. (eds.) (1990) Goodman and Gilman's: The Pharmacological Bases of Therapeutics, 8th Ed., Pergamon Press; Remington's Pharmaceutical Sciences, 17th ed. (1990), Mack Publishing Co., Easton, Pa.; Langer (1990) Science 249:1527-1533; Merck Index, Merck & Co., Rahway, N.J.; and Physician's Desk Reference (PDR).

The broad scope of this invention is best understood with reference to the following examples, which are not intended to limit the inventions to the specific embodiments.

### EXAMPLES

I. General Methods

Some of the standard methods are described or referenced, e.g., in Maniatis, et al. (1982) Molecular Cloning, A Laboratory Manual, Cold Spring Harbor Laboratory, Cold Spring Harbor Press; Sambrook, et al. (1989) Molecular Cloning: A Laboratory Manual, (2d ed.), vols. 1-3, CSH Press, NY; Ausubel, et al., Biology, Greene Publishing Associates, Brooklyn, N.Y.; or Ausubel, et al. (1987 and Supplements) Current Protocols in Molecular Biology, Greene/Wiley, New York. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification” in Meth. Enzymol., vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1990) “Purification of Recombinant Proteins with Metal Chelate Absorbent” in Setlow (ed.) Genetic Engineering, Principle and Methods 12:87-98, Plenum Press, N.Y.; and Crowe, et al. (1992) QlAexpress: The High Level Expression & Protein Purification System QIAGEN, Inc., Chatsworth, Calif.

Computer sequence analysis is performed, e.g., using available software programs, including those from the GCG (U. Wisconsin) and GenBank sources. Public sequence databases were also used, e.g., from GenBank and others.

II. Biopsy Samples

Six mm punch biopsies were taken, after obtaining informed consent, from either lesional skin of patients with psoriasis, atopic dermatitis, or cutaneous lupus erythematosus, or from normal healthy individuals. Skin samples were immediately frozen in liquid nitrogen and stored at −80° C. This study was performed under protocols approved by local ethics committees.

III. Cutaneous Wound Healing Model

Female BALB/c mice (8-12 weeks old) received a paravertebral full skin incision (2 cm) during anesthesia. 12 and 24 hours as well as 2, 3, 5, 7, and 10 days after initial injury, wounded skin was removed and used for RNA extraction, histology, immunohistochemistry, and collagen analyses.

IV. Cell Isolation and Cell Culture

Human primary epidermal keratinocytes, dermal fibroblasts, melanocytes, and dermal microvascular endothelial cells were purchased from Clonetics (San Diego, Calif.) and cultured in keratinocyte (KGM), fibroblast (FGM), melanocyte (MGM), or endothelial cell (EGM-2) growth medium (Clonetics, San Diego, Calif.). Cells were treated with TNF-α (10 ng/ml)/IL-1β (5 ng/ml), IFN-γ (20 ng/ml), IL-4 (50 ng/ml), IL-10 (100 ng/ml) (all R&D Systems Inc., Minneapolis, Minn.) or left untreated. The epidermal γδ T cell line, 7-17, was kindly provided by Richard Boismenu (The Scripps Institute, La Jolla, Calif.) and cultured. See Boismenu, et al. (1996) J. Immunol. 157:985-992. Epidermal γδ T cells were left untreated or stimulated with Con A, TNF-α (10 ng/ml)/IL-1β (5 ng/ml), for 6 or 18 h. Generation of dendritic cells either from cord blood CD34⁺ hematopoietic progenitor cells or from peripheral blood monocytes was performed. See Dieu, et al. (1998) J. Expt'l Med. 188:373-386. Human skin-derived Langerhans cells (LC) were isolated from normal skin of patients undergoing plastic surgery and enriched. See Dubois, et al. (1999) J. Immunol. 162:3428-3436. PBMCs were isolated using standard techniques and T cell enrichment was performed using T cell enrichment columns (R&D Systems Inc., Minneapolis, Minn.).

V. Analysis of Chemokine and Chemokine Receptor Expression

Skin biopsies and mouse skin samples were homogenized in liquid nitrogen using a Mikro-Dismembrator U (Braun Biotech, San Diego, Calif.) and RNA was extracted with RNAzol according to the manufacturer's protocol (Tel-Test, Friedensburg, Tex.). 4 μg of RNA were treated with DNase I (Boehringer, Mannheim, Germany) and reverse transcribed with oligo dT₁₄₋₁₈ (Gibco BRL, Gaithersburg, Md.) and random hexamer primers (Promega, Madison, Wis.) using standard protocols. cDNA was diluted to a final concentration of 5 ng/pl. 10 μl of cDNA were amplified in the presence of 12.5 μl of TaqMan® universal master mix (Perkin Elmer, Foster City, Calif.), 0.625 μof gene-specific TaqMan® probe, 0.5 μl of gene-specific forward and reverse primers, and 0.5 μl of water. As an internal positive control, 0.125 μl of 18S RNA-specific TaqMan® probe and 0.125 μl of 18S RNA-specific forward and reverse primers were added to each reaction. Specific primers and probes for the following human and mouse chemokines and chemokine receptors: CCR1 (CCR9), CXCR1 (CXCR5), XCR1, CX3CR1; MIP-1α (CCL3), MIP-1β (CCL4), MIP-18 (CCL15), MIP-3β (CCL19), 6Ckine (CCL21), IP-10 (CXCL10), MIG (CXCL9),I-309 (CCL1), I-TAC (CXCL11), HCC-1 (CCL14), HCC-4 (CCL16), Gro-α/62 (CXCL12), ENA78 (CXCL5), eotaxin (CCL11), eotaxin-2 (CCL24), DC-CK1 (CCL18), BCA-1 (CXCL13), fractalkine (CX3CL1), SDF-1α (CXCL12), RANTES (CCL5), PF4 (CXCL4), MDC (CCL22), lymphotactin (XCL1), IL-8 (CXCL8), TARC (CCL17), TECK (CCL25), and MCP-1 (CCL2), MCP-2 (CCL8), MCP-3 (CCL7), MCP4 (CCL13), were designed and validated, and obtained from Perkin Elmer (Foster City, Calif.). Gene-specific probes used FAM as reporter whereas probes for the internal positive control (18S RNA) were associated with either the JOE or VIC reporters. Samples underwent the following stages: stage 1, 50° C. for 2 min; stage 2, 95° C. for 10 min; and stage 3, 95° C. for 15 secs; followed by 60° C. for 1 min. Stage 3 was repeated 40 times. Gene-specific PCR products were measured by means of an ABI PRISM® 7700 Sequence Detection System (Perkin Elmer, Foster City, Calif.) continuously during 40 cycles. Specificity of primer probe combination was confirmed in crossreactivity studies performed against plasmids of all known chemokine receptors (CCR1-CCR9, CXCR1-CXCR5, XCR1, CX3CR1) and the following panel of chemokines: MIP-1α (CCL3), MIP-1β (CCL4), MIP-1β (CCL15), MIP-3β (CCL19), 6Ckine (CCL21), IP-10 (CXCL10), MIG (CXCL9),I-309 (CCL1), I-TAC (CXCL11), HCC-1 (CCL14), HCC-4 (CCL16), Gro-α/β (CXCL1/2), ENA78 (CXCL5), eotaxin (CCL11), eotaxin-2 (CCL24), DC-CK1 (CCL18), BCA-1 (CXCL13), fractalkine (CX3CL1), SDF-1α (CXCL12), RANTES (CCL5), PF4 (CXCL4), MDC (CCL22), lymphotactin (XCL1), IL-8 (CXCL8), TARC (CCL17), TECK (CCL25), and MCP-1 (CCL2), MCP-2 (CCL8), MCP-3 (CCL7), MCP4 (CCL13). Target gene expression was normalized between different samples based on the values of the expression of the internal positive control. Human cDNA libraries used in this study were generated. See, e.g., Rossi, et al. (1997) J. Immunol. 158:1033-1036; Bolin, et al. (1997) J. Neurosci. 17:5493-5502; and Vicari, et al. (1997) Immunity 7:291-301.

VI. Immunohistochemistry

Frozen 6 μm tissue sections were fixed in acetone and in paraformaldehyde before the immunostaining. To block non-specific binding of avidin, biotin system components, or endogenous peroxidase activity, sections were pre-treated with avidin D and biotin solutions (Blocking kit, Vector, Biosys SA, Compiegne, France) for 10 min each step and with PBS containing 0.3% hydrogen peroxide (Sigma, France) for 15 min at room temperature. After brief washing in PBS, the sections were incubated with blocking serum (2% normal rabbit serum) for at least 30 min before adding both primary antibodies. Sections were stained with anti-TARC, anti-IP-10, anti-MIG, anti-ITAC, anti-CXCR3, and anti-CXCR4 for 1 h at room temperature in a humid atmosphere. The binding of goat IgG was detected using biotinylated rabbit anti-goat IgG followed by streptavidin-peroxidase (both included in the Vectastain ABC kit: Goat IgG PK-4005, Vector) and the binding of mouse IgG1 was detected by rabbit alkaline phosphatase-labeled antimouse IgG (D0314, Dako, Glostrup, Denmark) at the same time at room temperature in a humid atmosphere. The peroxidase and alkaline phosphatase activities were revealed using 3-amino-9-ethylcarbazole (AEC) substrate (SK-4200, Vector) and alkaline phosphatase substrate III (SK-5300, Vector) for 5 to 10 min at room temperature, respectively. Negative controls were established by adding non-specific isotype controls as primary antibodies.

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

What is claimed is:

1. A method of diagnosing or evaluating a skin injury or condition affecting the skin, said method comprising evaluating expression of:
a) a chemokine selected from MCP-2 (CCL8), DC-CK1 (CCL18), TARC (CCL17), RANTES (CCL5), MIP3b (CCL19), I-309 (CCL1), MIG (CXCL9), IP-10 (CXCL10), ITAC (CXCL11), BCA-1 (CXCL13), lymphotactin (XCL1), MDC (CCL22), IL-8 (CXCL8), MCP-3 (CCL7), SDF-1, or MCP-1 (CCL2); or
b) a chemokine receptor selected from CCR5, CCR7, CXCR3, CXCR5, XCR1, CCR2, CCR4, CCR8, or CXCR4.

2. The method of claim 1, wherein said condition is selected from lupus erythematosus, atopic dermatitis, cutaneous wound, skin healing, or an inflammatory condition.

3. The method of claim 1, wherein said evaluating is:
a) measuring a plurality of said expression levels;
b) measuring mRNA levels; or
c) measuring protein levels.

4. A method of treating a condition affecting the skin, said method comprising administering an antagonist of:
a) a chemokine selected from MCP-2 (CCL8), DC-CK1 (CCL18), TARC (CCL17), RANTES (CCL5), MIP3b (CCL19), I-309 (CCL1), MIG (CCL9), IP-10 (CXCL10), ITAC (CXCL11), BCA-1 (CXCL13), lymphotactin (XCL1), MDC (CCL22), IL-8 (CXCL8), MCP-3 (CCL7), or MCP-1 (CCL2); or
b) a chemokine receptor selected from CCR5, CCR7, CXCR3, CXCR5, XCR1, CCR2, CCR4, CCR8, or CXCR4.

5. The method of claim 4, wherein said administering is:
a) a plurality of said antagonists; or
b) in combination with another therapeutic.

6. The method of claim 4, wherein said antagonist is an antibody which prevents interaction of:
a) said chemokine with its receptor, or
b) said chemokine receptor with its ligand.

7. The method of claim 4, wherein said treating is preventative.

8. The method of claim 4, wherein said condition is lupus erythematosus, and said antagonist is of:
a) a chemokine selected from MCP-2 (CCL8), RANTES (CCL5), MIP3b (CCL19), MIG (CXCL9), IP-10 (CXCL10); ITAC (CXCL11); BCA-1 (CXCL13), or lymphotactin (XCL1); or
b) a chemokine receptor selected from CCR5, CCR7, CXCR3, CXCR5, or XCR1.

9. The method of claim 4, wherein said condition is atopic dermatitis, and said antagonist is of:
a) a chemokine selected from DC-CK1 (CCL18), TARC (CCL17), I-309 (CCL1), MDC (CCL22), IP-10 (CXCL10), MIG (CXCL9), or ITAC (CXCL11); or
b) a CCR2, CCR3, CCR4, or CCR8 chemokine receptor.

10. A method of accelerating wound healing comprising administering to an individual suffering from a wound a chemokine selected from lymphotactin (XCL1), IL-8 (CXCL8), MCP3 (CCL7), MCP1 (CCL2), MCP2 (CCL8), RANTES (CCL5), MIG (CXCL9), or SDF-1.

11. The method claim 8, wherein said administering is:
a) a plurality of said chemokines;
b) in combination with another therapeutic; or
c) by expression of a nucleic acid.

12. The method of claim 8, wherein said healing is from skin loss from burn.
